(19) United States
(12) Patent Application Publication
(10) Pub. No.: US 2004/0143325 A1
(43) Pub. Date: Jul. 22, 2004

(54) Female nipple prosthetic device

(76) Inventor: Constance Tizard Holmes, New York, NY (US)

Correspondence Address: Steven Horowitz, Counselor At Law, Suite 700, 295 Madison Avenue, New York, NY, 10017, US

(21) Appl. No.: 10/349,449

(22) Filed: Jan. 22, 2003

(51) Int. Cl. A61F 2/52

(52) U.S. Cl. 623/7

(57) ABSTRACT

An artificial nipple prosthetic device attachable to a female human breast that has undergone a stage of breast reconstruction, comprising a substantially cylindrical nipple portion made of a breathable natural or microporous polymeric material. The nipple portion is solid to prevent collapse of the nipple portion. The device includes an areola portion surrounding the nipple portion made of the same breathable water-resistant natural material. The inside of the device, at both the nipple and areola portions, has a concave shape that conforms to an outer contour of a female human breast. The device is attached by means of a water resistant adhesive layer. Upon attachment of the nipple prosthetic device to the breast, the nipple portion projects outwardly from an outer surface of the female human breast, preferably substantially perpendicularly from said outer surface. The shades of color of the nipple and areola portions conform to the female anatomy.

### FIELD OF THE INVENTION

The field of this invention is prosthetic devices for humans, and more particularly, nipple prosthetic devices for the female human breast.

### BACKGROUND OF THE INVENTION AND DISCUSSION OF THE PRIOR ART

Medically, female breast reconstruction generally occurs in three stages. The first stage is the mastectomy. The second stage involves internal reconstruction of the breast. The third stage involves nipple reconstruction. During the period of time from the mastectomy to the nipple reconstruction the female patient can live without one or both nipples for a period of up to two years. If a pregnancy occurs, it could delay it even further.

Presently, the surgeon typically advises the patient perhaps a day before the nipple reconstruction surgery to draw a nipple where she wants it to be located and to thereby also indicate the size she desires it. That hardly provides the patient the time or the circumstances for appropriate psychological adjustment in order to make such a decision. It would be quite beneficial to have some way of greatly enhancing the psychological and practical factors surrounding the decision by the patient as to where, how large and what color her reconstructed nipple should be.

Furthermore, the clear majority of breast reconstruction patients never even reach the stage three reconstruction surgery simply due to the difficult psychological toll that the process takes. Accordingly, having some way of greatly enhancing the practical and psychological aspects of the decision by the patient as to where, how large and what color the reconstructed nipple should be would thus also have the incidental but crucial advantage of dramatically increasing the success rate of the entire three stage breast reconstruction procedure.

The present invention provides a female nipple prosthetic device to the patient for use during this period of time from the masectomy to the nipple reconstruction when the female patient is adjusting to breast reconstruction. As explained, the patient benefits greatly from having an artificial nipple prior to the actual nipple reconstruction since there is a need for such a device that is specifically designed to be worn by a female patient during the second stage of breast reconstruction.

During the second stage of breast reconstruction the patient's skin in the area of the nipple is traumatized and scarred from being aggravated by the implantation procedures of this second stage. Hence, the most appropriate female nipple prosthetic device would be one that causes no further irritability or aggravation to the skin in the affected area. This requirement means that the prosthetic device must be breathable and comfortable. Any prosthetic device against the skin that is nonporous is likely to lock in moisture from perspiration. This would causes discomfort and would aggravate and/or irritate the skin. In addition, it could reduce proper blood circulation in that area.

Furthermore, the fact that the period of use can be as long as two years and that the nipple can be worn for many days at a time, if not for entire weeks at a time, is an additional reason mandating that the artificial nipple be not only attractive and perceived as authentic but very comfortable also. In the past, artificial nipples have been made of plastic or other non-breathable materials since they were not designed specifically for a woman having breast reconstruction but rather as an effort to improve cosmetic appearance. See for example U.S. Pat. No. 5,171,321 to Davis. When worn for relatively short periods of time, plastic may seem to be an appropriate material for an artificial nipple since it is resilient and can be textured to achieve the tactile sensation that simulates a medically real nipple. A prosthetic device worn during breast reconstruction is something that is being placed on already scarred skin tissue which cannot withstand irritation. Accordingly, it must be suitable for such use. Furthermore, the prosthetic replaces a part of the human anatomy and should be structured so that it can be worn for lengthy periods of time if necessary yet be removable if such is desired. For such purposes, plastic and other non-breathable materials are not suitable.

Nipple prosthetic devices used for cosmetic purposes have been made of non-porous plastic and are not suitable for use during breast reconstruction. Even the plastic ones that have been fenestrated with one or more holes are not suitable either. Typically, the placement of a single hole occurs either in the center of the artificial nipple or in its surrounding area, such surrounding area being an artificial areola. It is questionable whether a single hole could alleviate the overall lack of breathability in the surface of the device that contacts the skin.

Furthermore, even a plastic nipple prosthetic device that is fenestrated with multiple holes would not be suitable for use during second stage breast reconstruction. The multiple holes (or for that matter even a single hole) would simply detract from the visual and tactile simulation purpose of the device. The purposes of having an artificial nipple is to simulate a biological nipple from a visual point of view and to simulate the biological nipple from a tactile point of view. Tactile simulation of a real nipple is necessary for any situation in which the nipple would likely be touched, such as during intimate contact. Having holes at various portions of the artificial nipple contradicts both the objective of visual simulation of the nipple as well as the objective of tactile simulation of the real nipple. A medically real nipple does not have holes, does not appear to have holes and not feel like it has holes.

It is also noted that women may be sensitive about the appearance of physical irregularities in their nipple and hence may even exaggerate the significance of an otherwise minor irregularity in the nipple. Accordingly, holes in the nipple, however minute, may be a disadvantage for a women.

Hence, these prior art nipple prosthetic devices used for cosmetic purposes are not suitable to be used as a nipple prosthetic device during breast reconstruction. There is a vital need for an artificial nipple that is both very comfortable to wear for long periods of time during breast reconstruction or otherwise and is as perfectly as possible identical to a biological female human nipple both visually and in terms of touch. Such an artificial nipple would have to be breathable without having holes that are perceptible either visually or through touch.

There is also a need for such a nipple prosthetic device that can be worn while taking a shower and/or while swimming. Further, there is a need for such a device that can be manufactured simply, preferably out of a single piece of material, and which can be offered in a variety of sizes, shapes and colors since different women may have different nipples in terms of size, shape and color and hence may prefer different artificial nipples. In order to finally decide on the desired size, location and color of the nipple the patient will receive during the third stage involving nipple reconstruction, it would be very helpful to be able to wear and try out for selected periods of time various nipple prosthetic devices that are different in terms of size, location and color. In this way, when the surgeon asks the patient to decide on the location, size and color of the nipple she desires, the patient will have had a meaningful opportunity to make that decision.

### SUMMARY OF THE PRESENT INVENTION

An artificial nipple prosthetic device attachable to a female human breast that has undergone breast reconstruction, comprising a substantially cylindrical nipple portion made of a breathable natural material. The nipple portion is solid to prevent collapse of the nipple portion. The device includes an areola portion surrounding the nipple portion made of the same breathable natural material. The inside of the device, at both the nipple and areola portions, has a concave shape that conforms to an outer contour of a female human breast. The device is attached by means of an adhesive layer that is water resistant. Upon attachment of the nipple prosthetic device to the breast, the nipple portion projects outwardly from an outer surface of the female human breast substantially perpendicularly from said outer surface. The color of the nipple and areola portions conform to an actual female anatomy and may display shaded gradations getting darker from the nipple to the areola portions.

### IMPORTANT OBJECTS AND ADVANTAGES

The following important objects and advantages of the present invention are:

(1) to provide a nipple prosthetic device for human females;

(2) to provide a nipple prosthetic device that is breathable;

(3) to provide a nipple prosthetic device that can be worn comfortably for days at a time,

(4) to provide a nipple prosthetic device that is appealing to women undergoing breast reconstruction;

(5) to provide a nipple prosthetic device that simulates a medically real female nipple both visually and in terms of tactile perception;

(6) to provide a nipple prosthetic device that simulates both the nipple and the surrounding areola;

(7) to provide a nipple prosthetic device that is made of breathable cotton or another natural breathable loosely woven fibrous material;

(8) to provide a nipple prosthetic device that in an alternative embodiment can be made from microporous polymeric material;

(9) to provide a nipple prosthetic device that is resilient;

(10) to provide a nipple prosthetic device that can be easily manufactured in a wide variety of sizes, shapes and colors;

(11) to provide a nipple prosthetic device that can be used by a woman undergoing breast reconstruction to try out the size, location and color of a nipple;

(12) to provide a nipple prosthetic device that is water resistant in that the device can be worn while showering or swimming without the device falling off from erosion of the adhesive layer; and

(13) to provide a nipple prosthetic device that can also be made use of by women who are interested in altering the appearance of their nipple with respect to size, shape and/or color.

### BRIEF DESCRIPTION OF THE DRAWINGS

FIG. 1 is a side elevational view of the device of the present invention attached to a female breast;

FIG. 2 is a cross-sectional view of the device of the present invention;

FIG. 3 is a top plan view of the present invention including the peelable backing.

### DETAILED DESCRIPTION OF THE PREFERRED EMBODIMENT

The apparatus of the present invention will now be illustrated by reference to the accompanying drawings. The prosthetic device of the present invention has been assigned reference numeral 10 Other elements have been assigned the reference numerals referred to below.

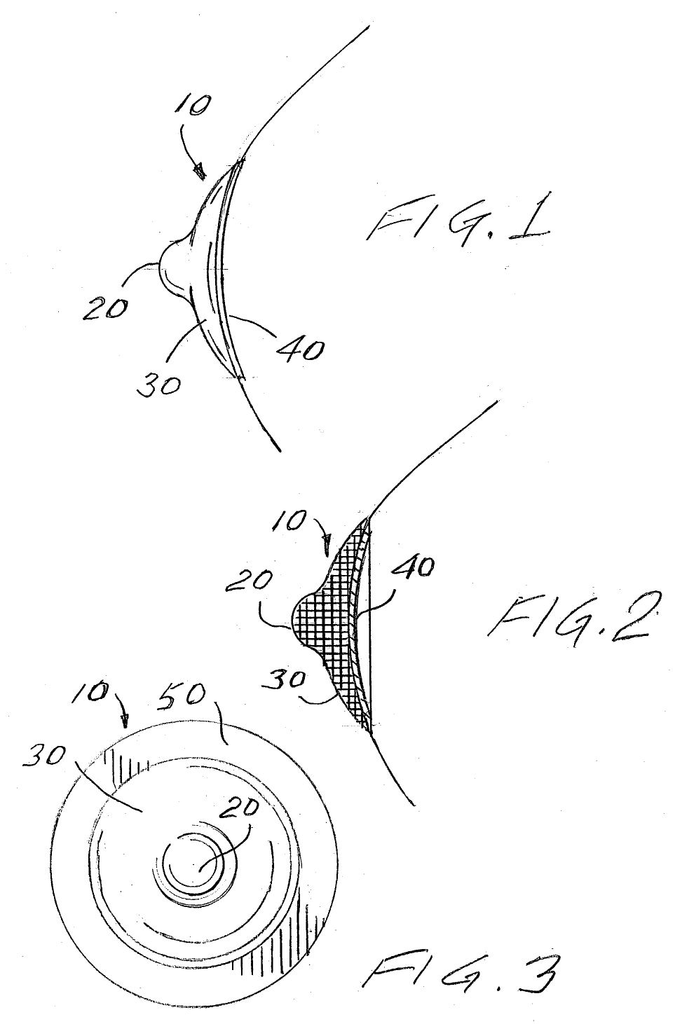

As seen from FIGS. 1-3, nipple prosthetic device 10 is shown as comprising a nipple portion 20 and an areola portion 30. Nipple prosthetic device 10 is thus attachable to a female human breast, usually one which has undergone breast reconstruction, by means of adhesive layer 40. Device 10 is stored against a peelable backing 50 (in embodiments where device 10 is defined to include backing 50 then the remainder of device 10 is stored against peelable backing 50). Typically, peelable backing 50 may be latex or other suitable material. Peelable backing 50 may be made of any material known to those skilled in the art to be suitable for removable contact with nipple portion 20 and areola portion 30, i.e. with breathable natural loosely woven fibrous material having a layer of adhesive under it. Peelable backing 50 allows the remainder of device 10 to be peeled off. Since the inside of device 10 is concave, peelable backing 50 is also concave. Alternatively, peelable backing would be in actual contact only with areola portion 30 or with an outer rim portion of areola portion 30.

As seen from FIGS. 1-2, nipple portion 20 and areola portion 30 may be described as substantially bell shaped when viewed together and from the side since said portions are shaped to replicate a biological female human breast nipple. Arguably, nipple portion 20 alone may also be described as substantially cylindrical overall.

Nipple portion 20 and areola portion 30 are formed from one continuous piece of material so these portions are made from the same material. Besides the shape, a difference between the nipple portion 20 and areola portion 30 is, however, observable in that there is a gradual change in color from lighter to darker as one goes from nipple portion 20 to areola portion 30. The preferred color of nipple portion 20 and areola portion 30 is the color of a natural female nipple and areola, which may be a shade of beige or yellow- reddish or skin color.

It is an important feature of the present invention that nipple portion 20 and areola portion 30 are made of a breathable material. Preferably, it is also a natural material to minimize or avoid allergic skin reactions. Furthermore, nipple portion 20 is not hollow but is solid so as to prevent collapse of nipple portion 20. Typically, the material from which nipple portion 20 and areola portion 30 are made is natural material rather than plastic or other man-made material. Preferably, the material used for nipple portion 20 and areola portion 30 should not be natural rubber, synthetic rubber, silicone or any plastic including but not limited to polyurethane, polyethylene, polypropylene or another synthetic polymer or plastic since these are not natural breathable materials. Rather the material should be cotton or another loosely woven fibrous material that is breathable. Ideally, the material should also be downy, i.e. soft.

In an alternative embodiment, the material from which nipple portion 20 and areola portion 30 are made is a water-resistant and breathable man-made material such as the polytetrafluorethylene disclosed in U.S. Pat. No. 4,187,390. Similarly, it is contemplated by the present invention that certain microporous man-made polymeric materials can be breathable and water-resistant and be usable as the material for the nipple portion 20 and areola portion 30.

It should be understood that the term “loosely woven” should exclude material that is so loosely woven that it either has holes that are visible or perceptible-through touch or that the resilience or durability of the material are compromised with respect to the purposes of the nipple prosthetic device of the present invention, namely accepting an adhesive layer and being able to adhere to the breast and stay there without being damaged, deformed or knocked off. The texture of the material used for nipple portion 20 and areola portion 30 should not be too loosely woven—device 10 must be sufficiently resilient and durable and maintain its shape in the face of forceful contact.

For example, certain kinds of loosely woven cotton are used to make gauze bandages and are breathable and suitable for nipple portion 20 and areola portion 30. Certain bandages contain the breathable material that represent examples of the desired material. Furthermore, such bandage materials are ideally suited to hold a layer of adhesive underneath that is water-resistant. Purely by way of example, an adhesive dressing bandage sold under the brand name “Coverlet” and which is manufactured by a company called Belersdorf-Jobst, Inc. of Rutherford College, North Carolina 28671 is believed to be breathable and water-resistant. The Coverlet brand bandages, though understood to be breathable, are dense enough to preclude the appearance of holes. This is in contrast to the breathable natural material of Sheer Band-Aid brand bandages sold by Johnson & Johnson, which though breathable has readily visible holes, or the Plastic Band-Aid brand bandages of Johnson & Johnson, which is not breathable. Other brands of breathable natural material or other breathable loosely woven fibrous material are well known to those skilled in the art and are available to satisfy the requirements of being breathable material yet having no visible holes.

However, since the breathable material from which nipple portion 20 and areola portion 30 are made must be shaped to duplicate the shape of a female nipple and areola, as shown in FIGS. 1-3, the thin layer used in a thin breathable bandage for wounds would alone not be suitable. Rather a thicker version of the same breathable material would have to be utilized and in addition shaped to conform to the shape of a female human breast nipple and the surrounding areola.

The areola portion 30 has a substantially round outer perimeter and surrounds nipple portion 20. Furthermore, the inside of device 10 has a concave shape at the inside surface of both nipple portion 20 and areola portion 30 so as to conform to an outer contour of a female human breast. It should be understood that nipple portion 20 includes a surface that is adjacent to adhesive layer 40 (in the embodiments where adhesive layer 40 is not limited to being in contact with areola portion 30). Upon attachment of the nipple prosthetic device 10 to the female breast, nipple portion 20 projects outwardly from an outer surface of the female human breast. In a preferred embodiment, nipple portion 20 projects substantially perpendicularly outward from said outer surface of said female human breast. By “substantially perpendicularly” is meant that an imaginary axis running longitudinally through a center of the nipple portion 20 would intersect a line tangent to the curved surface of the breast at an angle that deviates by not more than approximately twenty degrees from the normal.

Underneath nipple portion 20 and areola portion 30 is adhesive layer 40, a thin layer of adhesive whose effectiveness and/or adhering strength is specifically designed to be largely unaffected by water such that device 10 is water-resistant. Furthermore, nipple portion 20 and areola portion 30 remain breathable even though they are in contact with adhesive layer 40 either because the adhesive of adhesive layer 40 is not universally applied on the entire inside relevant surface of device 10 and/or because adhesive 40 is only lightly applied just enough to create non-permanent adherence without disturbing breathability and/or because of other reasons known to those skilled in the art.

In one embodiment, adhesive layer 40 covers an area that is adjacent to the entire inside surface of nipple portion 20 and areola portion 30. Alternatively, adhesive layer 40 is situated so as not to be in contact with nipple portion 20 at all (or in other alternative embodiments so as not to be in contact with all of nipple portion 20) but rather covers an area that is adjacent to and in contact with the inside concave surface of areola portion 30 and in fact contacts the entire such inside concave surface of areola portion 30. In a further alternative, adhesive layer 40 is situated so as to cover an area that is adjacent to and in contact with only a continuous portion of the inside surface of areola portion 30, and preferably a continuous annular segment of areola portion 40. In a further alternative embodiment, adhesive layer 40 is situated so as to cover an area that is adjacent to and in contact with only a noncontinuous segment of areola portion. In any of the alternative embodiments and in the preferred embodiment also, the adhesive layer 40 must cover a sufficient surface area so as to generate the required adhesion for device 10 to stay immediately adjacent the female reconstructed breast for long uninterrupted periods of time, including in or in exposure to water.

Furthermore, since the color, shape and size of female nipples vary, device 10 can be molded into different sizes and different colors. The different shapes of device 10 would arise from the nipple portion 20 of said device being shaped differently. The nipple portion 20 shown in FIGS. 1-2 is merely illustrative of one such shape. Device 10 would be available in a variety of shapes, sizes and colors.

In order to conform to the female anatomy, the nipple and areola portions are colored to conform to an actual female human nipple and areola and wherein a color of the nipple portion gradually changes to a darker color of the areola portion in an area of the areola portion closest to the nipple portion.

It is to be understood that while the apparatus of this invention have been described and illustrated in detail, the above-described embodiments are simply illustrative of the principles of the invention. It is to be understood also that various other modifications and changes may be devised by those skilled in the art which will embody the principles of the invention and fall within the spirit and scope thereof. It is not desired to limit the invention to the exact construction and operation shown and described. The spirit and scope of this invention are limited only by the spirit and scope of the following claims.

What is claimed is:

1. A breathable and water-resistant nipple prosthetic device attachable to a female human breast that has undergone breast reconstruction, comprising:
a substantially cylindrical nipple portion made of a breathable loosely woven fibrous material, said nipple portion being solid to prevent collapse of said nipple,
an areola portion surrounding said nipple portion, said areola portion having a concave shape that conforms to an outer contour of a female human breast, said areola portion made of a breathable loosely woven fibrous material,
said nipple portion aligned with said areola portion so that upon attachment of the nipple prosthetic device to the breast, said nipple portion projecting outwardly from an outer surface of the female human breast
a thin water resistant adhesive layer covers an area adjacent to and in contact with at least part of an inside surface of the areola portion sufficient to adhere the device to the female breast.
said nipple and areola portions being water resistant so that the nipple prosthetic device as a whole is water-resistant.

2. The nipple prosthetic device of claim 1, wherein the nipple portion and areola portion are made of cotton.

3. The nipple prosthetic device of claim 1, wherein the breathable loosely woven fibrous material is a natural material.

4. The nipple prosthetic device of claim 1, wherein the nipple portion projects substantially perpendicularly from said outer surface of said female human breast upon attachment to the breast.

5. The nipple prosthetic device of claim 1, wherein the nipple and areola portions are colored to conform to an actual female human nipple and areola and wherein a color of the nipple portion gradually changes to a color of the areola portion in an area of the areola portion closest to the nipple portion.

6. The nipple prosthetic device of claim 1, wherein the nipple portion projects substantially perpendicularly from said outer surface of said female human breast upon attachment to the breast, wherein the nipple and areola portions are colored to conform to an actual female human nipple and areola and wherein a color of the nipple portion gradually changes to a color of the areola portion in an area of the areola portion closest to the nipple portion.

7. The nipple prosthetic device of claim 1, wherein said device also includes a peelable backing that the adhesive layer of the device sticks to during storage

8. The nipple prosthetic device of claim 1, wherein the adhesive layer is adjacent to an entire inside surface of the areola portion.

9. The nipple prosthetic device of claim 8, wherein the adhesive layer is also adjacent to at least a part of an inside surface of the nipple portion.

10. The nipple prosthetic device of claim 8, wherein the adhesive layer is also adjacent to an entire inside surface of the areola portion and an entire inside surface of the nipple portion.

11. A breathable and water-resistant nipple prosthetic device attachable to a female human breast that has undergone breast reconstruction, comprising:
a substantially cylindrical nipple portion made of a breathable microporous water-resistant polymer material, said nipple portion being solid to prevent collapse of said nipple,
an areola portion surrounding said nipple portion, said areola portion having a concave shape that conforms to an outer contour of a female human breast, said areola portion made of a breathable microporous water-resistant polymer material,
said nipple portion aligned with said areola portion so that upon attachment of the nipple prosthetic device to the breast, said nipple portion projecting outwardly from an outer surface of the female human breast
a thin water resistant adhesive layer covers an area adjacent to and in contact with at least part of an inside surface of the areola portion sufficient to adhere the device to the female breast.

12. The nipple prosthetic device of claim 11, wherein the nipple portion projects substantially perpendicularly from said outer surface of said female human breast upon attachment to the breast.

13. The nipple prosthetic device of claim 11, wherein the nipple and areola portions are colored to conform to an actual female human nipple and areola and wherein a color of the nipple portion gradually changes to a color of the areola portion in an area of the areola portion closest to the nipple portion.

14. The nipple prosthetic device of claim 11, wherein the nipple portion projects substantially perpendicularly from said outer surface of said female human breast upon attachment to the breast, wherein the nipple and areola portions are colored to conform to an actual female human nipple and areola and wherein a color of the nipple portion gradually changes to a color of the areola portion in an area of the areola portion closest to the nipple portion.

15. The nipple prosthetic device of claim 11, wherein said device also includes a peelable backing that the adhesive layer of the device sticks to during storage

16. The nipple prosthetic device of claim 11, wherein the adhesive layer is adjacent to an entire inside surface of the areola portion.

17. The nipple prosthetic device of claim 16, wherein the adhesive layer is also adjacent to at least a part of an inside surface of the nipple portion.

18. The nipple prosthetic device of claim 16, wherein the adhesive layer is also adjacent to an entire inside surface of the areola portion and an entire inside surface of the nipple portion.
